(19) United States
(12) Patent Application Publication
(10) Pub. No.: US 2006/0165823 A1
(43) Pub. Date: Jul. 27, 2006

(54) Topical delivery agents and methods for making and using therm

(76) Inventor: Hector Herrera, Chula Vista, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/188,129

(22) Filed: Jul. 21, 2005

Related U.S. Application Data
(63) Continuation of application No. PCT/US04/03558, filed on Jan. 21, 2004.
(60) Provisional application No. 60/441,909, filed on Jan. 21, 2003.

(51) Int. Cl. A61K 36/48

(52) U.S. Cl. 424/757

(57) ABSTRACT

The invention provides methods for making and using topical delivery agents, wherein the delivery agents comprise an oil, e.g., jojoba oil, and an active agent, and formulations made by the methods of the invention.

### TECHNICAL FIELD

This invention relates to the field of pharmaceuticals and drug delivery, and more particularly, to methods for making and using topical delivery agents, wherein the delivery agents comprise an oil, e.g., jojoba oil, and an active agent. The invention also provides formulations made by the methods of the invention.

### BACKGROUND

The topical application of active compounds to the skin forms the basis of most dermatological therapy. This application can have advantages, such as absence of pain and of bad taste, simplicity and ease of administration, and high concentration at the desired site. It is a method by which a high local concentration of drugs in the skin can be achieved without undesirable systemic side effects. In many instances, it would be advantageous if the rate of penetration of active compounds through the epidermis could be increased. This would help in bringing about a more rapid and profound action of the locally applied compounds. If penetration, e.g., transfollicular, transepidermal and into the stratum corneum, could be increased, many new compounds could be introduced into therapy.

Active compounds may traverse the skin primarily through the opening of the hair follicles, the sweat gland ducts or by passing through the protein/lipid domains of the stratum corneum. From the skin surface, the active compounds may subsequently diffuse into the intracellular spaces and the cell. In the initial transient diffusion stage, penetration may occur through the skin appendages, i.e. the hair follicles and the ducts. It can then pass into the skin.

Many substances show enhanced absorption through the skin when dissolved in water, propylene glycol, butylene glycol, polyethylene glycol, ethanol, dimethyl sulfoxide and other polar and non-polar solvents. The vehicle generally does not increase the rate of penetration into the skin, but serves as a carrier. Insoluble compounds must be uniformly dispersed throughout the vehicle to assure homogeneity of the product. Milling to a finely divided state may provide more surface area for contact with the dermal site and increases penetration through the intercellular spaces of the skin structures.

The major factors that determine the penetrating ability of a substance into the skin include its molecule size and its lipophilicity. Beyond a certain size, molecules cannot penetrate the skin. Only relatively small molecules can penetrate the skin. For example, collagen, which is present in many cosmetic products, has relatively large molecules that cannot penetrate the skin. Oily products may penetrate the skin more easily than water-based preparations. Substances with better oil solubility (more lipophilic) may penetrate the skin more easily than water-based preparations.

The hair follicle, hair shaft and sebaceous gland are collectively known as the pilosebaceous unit. The pilosebaceous unit is a complex, dynamic, 3D structure, which is the site for unique biochemical, metabolic and immunological events. Ongoing research has focused on the pilosebaceous unit as a potential route for both localized and systemic drug delivery. Targeted drug delivery to the specific sites of hair follicle has been used to treat several dermatological conditions that are known to originate at the hair follicle.

Dermatology patients and cosmetic consumers apply a wide span of topical preparations to their healthy or diseased skin. These preparations have physico-chemical natures that range from simple liquids to semisolids to powders and transdermal therapeutic systems. Topical preparations may include a vehicle that releases an active agent for optimum absorption. Topical preparations may include lubricant and emollient effects, cleansing and protection effects, symptomatic relief of itch and pain or anti-inflammatory effects, as in acute inflammation.

One of the problems associated with delivery of active compounds through the skin is volatility of the carrier. Many pharmaceutical and cosmetic compositions utilize ethanol as the delivery agent. Upon contact with the skin, alcohol evaporates and leaves the active compound on the surface of the skin.

Secretions of sebaceous glands throughout the skin create an oily layer that protects the skin from moisture loss and irritation, provides a protective barrier, lubricates, softens and has anti-microbial activity. This oily layer also hinders delivery of hydrophilic substances into the skin.

Insoluble active compounds incorporated in hydrophilic carriers can be difficult to deliver into the skin. To address this problem, insoluble active compounds have been incorporated in a carrier in the form of finely milled particles. However, the stratum corneum may remain a barrier to finely milled particles. Commercially available products containing benzoyl peroxide for treatment of acne are a good example. Because penetration of active agent into the skin is very limited, very high concentrations (up to 10%) have been utilized, thereby exposing the skin to high concentrations of irritating and drying compounds.

Sebum is a fatty substance secreted by the sebaceous glands of the skin. Most of these glands open into hair follicles. Hair follicles constitute entryways into the skin traversing the epidermis into the dermis. If an active compound is to be delivered to the skin through the hair follicle its carrier miscibility in sebum is a consideration.

### SUMMARY

The invention provides methods and agents for selectively delivering compounds to the skin and mucosa, including, e.g., to pilosebaceous units, such as hair follicles and sebaceous glands. The invention provides topical formulations comprising an active agent (e.g., a drug) and an oil, e.g., an oil made substantially of straight chain esters of mono-unsaturated long chain fatty acids and fatty alcohols having an average total carbon chain length of about 42 carbons, e.g., jojoba oil. The method and agents of the invention are effective for the topical or mucosal delivery of pharmaceuticals, cosmetics, vitamins and other nutritional or therapeutic agents.

In the compositions and methods of the invention, including topical formulations and articles of manufacture, the formulation can be made by a method comprising the following steps: (a) providing an active compound and a jojoba oil; and (b) mixing the active compound and the jojoba oil at room temperature without the use of heat until complete dissolution of the compound. In one aspect, room temperature is between about 20° C. to 25° C.

In the compositions and methods of the invention, including topical formulations and articles of manufacture, the formulation can be made by a method comprising the following steps: (a) providing an active compound and a jojoba oil; and (b) mixing the active compound and the jojoba oil with heat until complete dissolution of the compound. In one aspect, the active compound and the jojoba oil are mixed at temperatures above about 25° C., at temperatures above about 35° C., 45° C., 55° C., 65° C., 75° C., 85° C. or 95° C., or, at temperatures at about 10° C. In one aspect, if the formulation comprises benzoyl peroxide as an active compound, the temperature of the mixture should not exceed about 40° C.

In one aspect, the active compound comprises, based on the total weight of the formulation, from about 0.01 percent to about 5 percent, or, from about 0.1 percent to 4 percent, or, from about 1 percent to 4 percent, or, from about 2 percent to 4 percent of active compound.

In the compositions and methods of the invention, including topical formulations and articles of manufacture, the formulation can be made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution. In one aspect, the solubility enhancing agent is selected from the group consisting of an ethanol, a polyol or a mixture thereof. The polyol can be propylene glycol, glycerol and/or polyethylene glycol. In one aspect, the solution is an aqueous solution.

In the compositions and methods of the invention, including topical formulations and articles of manufacture, the formulations can comprise a fine particle dispersion of an active compound in jojoba oil, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil. In one aspect, the mixture is accelerated to pressures ranging from about 1,000 to about 30,000 psi, or, from about 3,000 to about 23,000 psi. In one aspect, the mixing is done in a microfluidizer-homogenizer. In one aspect the mixture is stirred at speeds ranging from about 1,000 rpm to 25,000 rpm, or from 5,000 rpm to 15,000 rpm. In one aspect the mixing is done in a rotor-stator homogenizer. In one aspect, the high shear rates produce micron-sized particles, or, the high shear rates produce sub-micron-sized particles. The active compound and the jojoba oil can be mixed by stirring or by high pressure homogenizing.

The formulation in the compositions and methods of the invention can further comprise a pharmaceutically acceptable excipient. The jojoba oil can be derived from a natural source or can be synthetic (e.g., derived from a synthetic source). In one aspect, the jojoba oil substantially comprises straight chain esters of mono-unsaturated long chain fatty acids and fatty alcohols having an average total carbon chain length of about 42 carbons. In alternative aspects, the fatty alcohols having an average total carbon chain length of about 20 to 60 carbons, about 30 to 50 carbons, about 35 to 45 carbons or about 40 to 44 carbons. In alternative aspects, the “substantially” is 100%, or about 99%, 98%, 97%, 96%, 95%, 90%, 85%, i.e., oil made substantially of about is 100%, 99%, 98%, 97%, 96%, 95%, 90%, 85% straight chain esters of mono-unsaturated long chain fatty acids and fatty alcohols having an average total carbon chain length of about 42 carbons, or equivalent thereof.

In one aspect, the jojoba oil is a pure, natural, golden grade oil; a refined and bleached grade oil; a decolorized and deodorized grade oil; a molecular distilled grade oil or a combination thereof. The formulation can comprise between about 5% and 99% jojoba oil, between about 10% and 90% jojoba oil, between about 20% and 80% jojoba oil. The formulation can comprise an emulsion, a paste, a gel, a cream, a lotion, an aqueous solution, a foam or an ointment. In one aspect, the formulation comprises a spray or a stick.

In the compositions and the methods of the invention, the active agent can be a pharmaceutical agent, a cosmetic, a nutritional supplement or a vitamin. In one aspect, the formulation further comprises an antioxidant. The antioxidant can comprise a concentration of at least about 60 ppm, 50 ppm or 40 ppm. The antioxidant can comprise an alpha-tocopherol, a gamma-tocopherol, a delta-tocopherol or a combination thereof. The active compound can comprise an antibiotic, such as erythromycin, tetracycline, minocycline, neomycin, penicillin or mixtures thereof. The active compound can comprise a plant part or plant extract, such as aloe vera, lavender, chamomile, calendula, Echinacea, saw palmetto, green tea, gingko biloba, birch, kiwi, magnolia, peppermint, philodendron or mixtures thereof. In one aspect, the part or plant extract comprises green tea, mulberry, genistein, daidzein from soy, any soy extract or product, or a mixture thereof.

In one aspect, the active compound comprises an antifungal, such as clotrimazole, tolnaftate, terbinafine hydrochloride or mixtures thereof. In one aspect, the active compound comprises an analgesic or an anesthetic, such as benzocaine, menthol, phenol, camphor, methyl salicylate or mixtures thereof. In one aspect, the active compound comprises an anti-aging agent, such as vitamin E, vitamin A, vitamin C, vitamin B and derivatives thereof; a retinoid; an antioxidant; a plant extract or mixtures thereof. In one aspect, the antioxidant comprises an alpha hydroxy acid, such as lactic acid (e.g., DL lactic acid), glycolic acid, citric acid, malic acid, ascorbic acid, tartaric acid, or a combination thereof. In one aspect, the antioxidant comprises a beta hydroxy acid, such as beta hydroxybutyric acid, beta phenyl lactic acid, DL lactic acid or a combination thereof.

In one aspect, the active compound comprises an anti-acne agent, such as benzoyl peroxide, salicylic acid, a topical retinoid, a tetracycline, an erythromycin or a mixture thereof. In one aspect, the topical retinoid comprises retinol, tretionin, adapalene, isotretionin, azelaic acid, motretinide, tazarotene or a mixture thereof.

In one aspect, the active compound comprises a hair growth promoter, such as rogaine, minoxidil (e.g., LONITEN®, UpJohn), Serenoa repens (saw palmetto) extract, finasteride, PROPECIA™, or a mixture thereof. In one aspect, the active compound comprises an anti-dandruff agent, an anti-psoriasis agent, an anti-seborrheic agent or an anti-dermatitis agent. The active compound can also comprise coal tar, selenium sulfide, sulfur, zinc pyrithione, salicylic acid, ketoconazole, clotrimazole, miconazole, fluconazole, vitamin A analogs, corticosteroids or mixtures thereof. The active compound can also comprise vitamin B complex, vitamin A, vitamin C, vitamin D, vitamin E, vitamin K, vitamin derivatives or mixtures thereof. The vitamin B complex can comprise thiamine, biotin, riboflavin, vitamin B6 or vitamin B12.

In one aspect, the active compound comprises an aminoacid, e.g., a synthetic amino acid or any of the 20 natural amino acids, including cysteine, glutamic acid, glycine, alanine, serine, valine, methionine, tryptophan, leucine and mixtures thereof.

The invention provides articles of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; and (b) mixing the active compound and the jojoba oil at room temperature without the use of heat until complete dissolution of the compound. In one aspect, room temperature is between about 20° C. to 25° C.

The invention provides articles of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; and (b) mixing the active compound and the jojoba oil with heat until complete dissolution of the compound. In alternative aspects, the active compound and the jojoba oil are mixed at temperatures above about 25° C., mixed at temperatures above about 35° C., 45° C., 55° C., 65° C., 75° C., 85° C. or 95° C., or mixed at temperatures at about 100° C.

The invention provides articles of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution. In one aspect, the solubility enhancing agent is selected from the group consisting of an ethanol, a polyol or a mixture thereof. The polyol can be propylene glycol, glycerol and/or polyethylene glycol. In one aspect, the solution is an aqueous solution.

The invention provides articles of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil. In one aspect, the mixture is accelerated to pressures ranging from about 1,000 to about 30,000 psi, or, from about 3,000 to about 23,000 psi. In one aspect, the mixing is done in a microfluidizer-homogenizer. In one aspect the mixture is stirred at speeds ranging from about 1,000 rpm to 25,000 rpm, or from 5,000 rpm to 15,000 rpm. In one aspect the mixing is done in a rotor-stator homogenizer. In one aspect, the high shear rates produce micron-sized particles, or, the high shear rates produce sub-micron-sized particles. The active compound and the jojoba oil can be mixed by stirring or by high pressure homogenizing.

In the compositions and methods of the invention, including the formulations and articles of manufacture, the formulation can further comprise a pharmaceutically acceptable excipient. The jojoba oil can be derived from a natural source or can be synthetic (e.g., derived from a synthetic source). In one aspect, the jojoba oil substantially comprises straight chain esters of mono-unsaturated long chain fatty acids and fatty alcohols having an average total carbon chain length of about 42 carbons. In alternative aspects, the fatty alcohols having an average total carbon chain length of about 20 to 60 carbons, about 30 to 50 carbons, about 35 to 45 carbons or about 40 to 44 carbons. In alternative aspects, the “substantially” is 100%, or about 99%, 98%, 97%, 96%, 95%, 90%, 85%, i.e., oil made substantially of about is 100%, 99%, 98%, 97%, 96%, 95%, 90%, 85% straight chain esters of mono-unsaturated long chain fatty acids and fatty alcohols having an average total carbon chain length of about 42 carbons, or equivalent thereof.

In one aspect, the jojoba oil is a pure, natural, golden grade oil; a refined and bleached grade oil; a decolorized and deodorized grade oil; a molecular distilled grade oil or a combination thereof. The formulation can comprise between about 5% and 99% jojoba oil, between about 10% and 90% jojoba oil, between about 20% and 80% jojoba oil. The formulation can comprise an emulsion, a paste, a gel, a cream, a lotion, an aqueous solution, a foam or an ointment. In one aspect, the formulation comprises a spray or a stick. The formulation can comprise an emulsion, a paste, a gel, a cream, a lotion, an aqueous solution, a foam or an ointment. In one aspect, the formulation comprises a spray or a stick.

In the compositions and methods of the invention, including the formulations and articles of manufacture, the active agent can be a pharmaceutical agent, a cosmetic, a nutritional supplement or a vitamin. In one aspect, the formulation further comprises an antioxidant. The antioxidant can comprise a concentration of at least about 60 ppm, 50 ppm or ppm. The antioxidant can comprise an alpha-tocopherol, a gamma-tocopherol, a delta-tocopherol or a combination thereof. The active compound can comprise an antibiotic, such as erythromycin, tetracycline, minocycline, neomycin, penicillin or mixtures thereof. The active compound can comprise a plant part or plant extract, such as aloe vera, lavender, chamomile, calendula, Echinacea, saw palmetto, green tea, gingko biloba, birch, kiwi, magnolia, peppermint, philodendron or mixtures thereof. In one aspect, the part or plant extract comprises green tea, mulberry, genistein, daidzein (e.g., from soy), any soy extract or product, or a mixture thereof.

In one aspect, the active compound comprises an antifungal, such as clotrimazole, tolnaftate, terbinafine hydrochloride or mixtures thereof. In one aspect, the active compound comprises an analgesic or an anesthetic, such as benzocaine, menthol, phenol, camphor, methyl salicylate or mixtures thereof. In one aspect, the active compound comprises an anti-aging agent, such as vitamin E, vitamin A, vitamin C, vitamin B and derivatives thereof, a retinoid; an antioxidant; a plant extract or mixtures thereof. In one aspect, the antioxidant comprises an alpha hydroxy acid, such as lactic acid (e.g., DL lactic acid), glycolic acid, citric acid, malic acid, ascorbic acid, tartaric acid, or a combination thereof. In one aspect, the antioxidant comprises a beta hydroxy acid, such as beta hydroxybutyric acid, beta phenyl lactic acid, DL lactic acid or a combination thereof.

In one aspect, the active compound comprises an anti-acne agent, such as benzoyl peroxide, salicylic acid, a topical retinoid, a tetracycline, an erythromycin or a mixture thereof. In one aspect, the topical retinoid comprises retinol, tretionin, adapalene, isotretionin, azelaic acid, motretinide, tazarotene or a mixture thereof.

In one aspect, the active compound comprises a hair growth promoter, such as rogaine, minoxidil, Serenoa repens (saw palmetto) extract, finasteride, PROPECIA™ or a mixture thereof. In one aspect, the active compound comprises an anti-dandruff agent, an anti-psoriasis agent, an anti-seborrheic agent or an anti-dermatitis agent. The active compound can also comprise coal tar, selenium sulfide, sulfur, zinc pyrithione, salicylic acid, ketoconazole, clotrimazole, miconazole, fluconazole, vitamin A analogs, corticosteroids or mixtures thereof. The active compound can also comprise vitamin B complex, vitamin A, vitamin C, vitamin D, vitamin E, vitamin K, vitamin derivatives or mixtures thereof. The vitamin B complex can comprise thiamine, biotin, riboflavin, vitamin B6 or vitamin B12. In one aspect, the active compound comprises an aminoacid, e.g., a synthetic amino acid or any of the 20 natural amino acids, including cysteine, glutamic acid, glycine, alanine, serine, valine, methionine, tryptophan, leucine and mixtures thereof.

The invention provides methods for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil by stirring at room temperature without the use of heat until complete dissolution of the compound. In one aspect, the room temperature is between about 20° C. to 25° C.

The invention provides methods for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil with heat until complete dissolution of the compound. The active compound and the oil, e.g., jojoba oil, can be mixed at temperatures above about 25° C., about 35° C., 45° C., 55° C., 65° C., 75° C., 85° C. or 95° C., or at about 100° C.

The invention provides methods for delivering an active compound into a hair follicle, a sebaceous gland or into skin comprising topical application to a subject of a formulation comprising an active compound and a jojoba oil, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution. The solubility enhancing agent can be an ethanol, a polyol or a mixture thereof. The polyol can be propylene glycol, glycerol and/or polyethylene glycol. The solution can be an aqueous solution.

The invention provides methods for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil.

In alternative aspects, the mixture is accelerated to pressures ranging from about 1,000 to about 30,000 psi, or, from about 3,000 to about 23,000 psi. In one aspect, the mixing is done in a microfluidizer-homogenizer. In one aspect the mixture is stirred at speeds ranging from about 1,000 rpm to 25,000 rpm, or from 5,000 rpm to 15,000 rpm. In one aspect the mixing is done in a rotor-stator homogenizer. In one aspect, the high shear rates produce micron-sized particles, or, the high shear rates produce sub-micron-sized particles. The active compound and the jojoba oil can be mixed by stirring or by high pressure homogenizing.

In practicing the methods of the invention, and in applying the compositions (e.g., formulations) of the invention, the subject can be any animal, e.g., a mammal, such as a human.

The invention provides compositions and methods for delivering a compound into the stratum corneum comprising topical application to a subject a formulation comprising the compound and a jojoba oil, wherein the formulation is made by a method comprising the following steps: (a) providing an active compound and a jojoba oil; (b) mixing the active compound and the jojoba oil by stirring at room temperature without the use of heat until complete dissolution of the compound. The active compound and the oil, e.g., jojoba oil, can be mixed at temperatures above about 25° C., about 35° C., 45° C., 55° C., 65° C., 75° C.; 85° C. or 95° C., or at about 100° C.

The invention provides compositions and methods for delivering a compound into the stratum corneum comprising topical application to a subject a formulation comprising the compound and a jojoba oil, wherein the formulation is made by a method comprising the following steps: (a) providing an active compound and a jojoba oil; (b) mixing the active compound and the jojoba oil with heat until complete dissolution of the compound. The active compound and the oil, e.g., jojoba oil, can be mixed at temperatures above about 25° C., above about 30° C., 35° C., 40° C., 45° C., 50° C., 55° C., 60° C., 65° C., 70° C., 75° C., 80° C., 85° C., 90° C., or 95° C., 96° C., 97° C., 98° C., 99° C., or at about 100° C.

The invention provides compositions and methods for delivering a compound into the stratum corneum comprising topical application to a subject of a formulation comprising the compound and a jojoba oil, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution.

The solubility enhancing agent can be an ethanol, a polyol or a mixture thereof. The polyol can be propylene glycol, glycerol and/or polyethylene glycol. The solution can be an aqueous solution.

The invention provides compositions and methods for delivering a compound into the stratum corneum comprising topical application to a subject of a formulation comprising the compound and a jojoba oil, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil.

In alternative aspects, the mixture is accelerated to pressures ranging from about 1,000 to about 30,000 psi, or, from about 3,000 to about 23,000 psi. In one aspect, the mixing is done in a microfluidizer-homogenizer. In one aspect the mixture is stirred at speeds ranging from about 1,000 rpm to 25,000 rpm, or from 5,000 rpm to 15,000 rpm. In one aspect the mixing is done in a rotor-stator homogenizer. In one aspect, the high shear rates produce micron-sized particles, or, the high shear rates produce sub-micron-sized particles. The active compound and the jojoba oil can be mixed by stirring or by high pressure homogenizing.

The invention provides compositions and methods comprising a hair growth promoter and jojoba oil, wherein the formulation is made by the method of claim 1 to claim 15, and the formulation comprises, based on the total weight of the formulation, from about 0.005 percent to about 5 percent, or, about 0.01 percent to about 3 percent, or, from about 0.1 percent to 2.5 percent, or, from about 1 percent to 2 percent of hair growth promoter. The hair growth promoter can comprise rogaine, minoxidil, Serenoa repens (saw palmetto) extract, finasteride, PROPECIA™ or a mixture thereof.

The invention provides topical formulations and method for making these compositions comprising an acne ameliorating agent and jojoba oil, wherein the formulation is made by the method of claim 1 to claim 15, and the formulation comprises, based on the total weight of the formulation, from about 0.005 percent to about 5 percent, or, about 0.01 percent to about 3 percent, or, from about 0.1 percent to 2.5 percent, or, from about 1 percent to 2 percent of acne ameliorating agent. The acne ameliorating agent can comprise benzoyl peroxide.

The invention provides methods for making formulations (and the compositions made by these methods) comprising an emulsion of an insoluble compound in a jojoba oil comprising mixing the insoluble compound in a jojoba oil and accelerating the mixture to high velocities, thereby subjecting the mixture to high shear rates to produce an emulsion of uniform droplets. In one aspect, the mixing is done in a high pressure microfluidizer-homogenizer, such as a M-110F Microfluidizer, available from Microfluidics Corp., Newton, Mass. In one aspect, the process pressures range from about 1,000 to about 25,000 psi, about 3,000 to about 23,000 psi or about 5,000 to about 20,000 psi. In one aspect the mixture is done in a rotor-stator homogenizer, such as a L4RT High Shear Mixer, available from Silverson Machines, Inc., East Longmeadow, Mass. In one aspect the mixture is stirred at speeds ranging from about 1,000 rpm to 25,000 rpm, or from 5,000 rpm to 15,000 rpm. The insoluble compound can comprise a liquid, such as a liquid comprising a drug, a solubilizing agent, a vitamin, an antioxidant, e.g., DL lactic acid, and the like. In one aspect, micron-sized droplets are produced, or sub-micron-sized droplets are produced. In one aspect, the insoluble compound comprises, based on the total weight of the formulation, from about 0.01 percent to about 5 percent, or, from about 0.1 percent to 4 percent, or, from about 1 percent to 4 percent, or, from about 2 percent to 4 percent of insoluble compound. The invention provides formulations made by the method methods of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A and 1B illustrate histological views of the dorsal skin of Sprague Dawley rat treated with a solution of 0.125% Nile Red in Jojoba Oil, unstained frozen sections, 50 micron thick, Light microscopy. FIGS. 1C and 1D illustrate corresponding histological views to FIGS. 1A and 1B observed under fluorescence microscopy. Experiments described in detail in Examples below.

FIGS. 2A and 2B illustrate histological views of the dorsal skin of Sprague Dawley rat treated with a solution of 0.125% Nile Red in Diethylene Glycol Monoethyl Ether (Transcutol, Lipscomb Chemical Company, Inc., Long Beach, Calif.), unstained frozen sections, 50 micron thick, Light microscopy. FIGS. 2C and 2D illustrate corresponding histological views to FIGS. 2A and 2B observed under fluorescence microscopy. Experiments described in detail in Examples below.

FIGS. 3A and 3B illustrate histological views of the dorsal skin of Sprague Dawley rat treated with a solution of 0.125% Nile Red in Dimethyl Sulfoxide (DMSO), unstained frozen sections, 50 micron thick, Light microscopy. FIGS. 3C and 3D illustrate corresponding histological views to FIGS. 3A and 3B observed under fluorescence microscopy. Experiments described in detail in Examples below.

FIGS. 4A and 4B illustrate histological views of the dorsal skin of Sprague Dawley rat treated with a solution of 0.125% Nile Red in a solution containing 50% Propylene glycol, 30% Ethanol and 20% Water, unstained frozen sections, 50 micron thick, Light microscopy. FIGS. 4C and 4D illustrate corresponding histological views to FIGS. 4A and 4B observed under fluorescence microscopy. Experiments described in detail in Examples below.

FIGS. 5A and 5B illustrate histological views of untreated dorsal skin of Sprague Dawley rat, unstained frozen sections, 50 micron thick, Light microscopy. FIGS. 5C and 5D illustrate corresponding histological views to FIGS. 5A and 5B observed under fluorescence microscopy. Experiments described in detail in Examples below.

Like reference symbols in the various drawings indicate like elements.

### DETAILED DESCRIPTION

In one aspect, the invention provides delivery agents and methods for selective delivery of compounds to the skin and mucosa. In one aspect, the invention provides delivery agents and methods for selective delivery of compounds to the pilosebaceous unit. The invention provides formulations comprising an active agent (e.g., a drug, vitamin and the like) and an oil made substantially of straight chain esters of mono-unsaturated long chain fatty acids and fatty alcohols having an average total carbon chain length of about 42 carbons, e.g., jojoba oil. The compositions and methods are effective for the topical or mucosal delivery of pharmaceuticals, cosmetics, vitamins and other nutritional or therapeutic agents.

In one aspect, the present invention describes a new method for transfollicular delivery of active compounds to the skin or mucosa, e.g., to the pilosebaceous units, using and oil made substantially of straight chain esters of mono-unsaturated long chain fatty acids and fatty alcohols having an average total carbon chain length of about 42 carbons, e.g., jojoba oil as the delivery agent. Compounds can be incorporated into the oil by dissolution with or without the aid of heat or solubility enhancers, by emulsification or by suspension.

In one aspect, the oil, e.g., the jojoba oil, is completely miscible in sebum and readily penetrates the pilosebaceous unit. This results in the need for lower concentrations of an active compound in the topical formulation to achieve the desired effect. Jojoba Oil can be very stable and resistant to oxidation, therefore, in one aspect, the topical formulation of the invention has a long shelf life and requires no special storage conditions. In one aspect, the topical formulation of the invention is non-toxic and presents a very low viscosity, easily spreading over the skin without the need for rubbing or massaging. In one aspect, the jojoba oil is a natural emollient, softening and helping retain water in the skin and preventing the irritating or drying effects of certain active compounds (i.e. benzoyl peroxide, alpha hydroxy acids, etc.). In one aspect, to effectively deliver active compounds through the hair follicle, the physicochemical nature of jojoba oil is maintained to preserve its properties, particular its miscibility with sebum. In one aspect, active compounds to be delivered are incorporated in the jojoba oil and remain incorporated until delivered to the target skin structures.

While the invention is not limited to any particular mechanism of action, experiments conducted using the compositions and methods of the invention have demonstrated that jojoba oil can penetrate the skin through the opening of the hair follicle and distribute itself throughout the hair shaft and sebaceous gland. In one aspect, pure jojoba oil penetrates deeper and targets the pilosebaceous unit.

Jojoba Oil

As used herein the term “Jojoba Oil” includes the oil extracted from seeds of the Jojoba shrub (Simmondsia chinensis) or any oil made substantially of straight chain esters of mono-unsaturated long chain fatty acids and fatty alcohols having an average total carbon chain length of about 42 carbons, or equivalent thereof. In alternative aspects, the fatty alcohols having an average total carbon chain length of about 30 to 50 carbons, about 35 to 45 carbons or about 40 to 44 carbons. In alternative aspects, the “substantially” is 100%, or about 99%, 98%, 97%, 96%, 95%, 90%, 85%, i.e., oil made substantially of about is 100%, 99%, 98%, 97%, 96%, 95%, 90%, 85% straight chain esters of mono-unsaturated long chain fatty acids and fatty alcohols having an average total carbon chain length of about 42 carbons, or equivalent thereof.

In one aspect, the oil made substantially of straight chain esters of mono-unsaturated long chain fatty acids and fatty alcohols having an average total carbon chain length of about 42 carbons, e.g., jojoba oil, used in the compositions and methods of the invention is completely miscible in human sebum. Its esters can be similar to the esters that make up 25 to 30% of human sebum. In alternative aspects, the oil made substantially of straight chain esters of mono-unsaturated long chain fatty acids and fatty alcohols having an average total carbon chain length of about 42 carbons, e.g., jojoba oil, used in the compositions and methods of the invention have low viscosity, are non-greasy, have resistance to oxidation, are non-toxic, do not support microbial growth, are emollients and have long shelf life. While the invention is not limited to any particular mechanism of action, in one aspect, the oils used in the compositions and methods of the invention acts as an delivery agent for active compounds into the skin via the hair follicle.

Jojoba Oil can be extracted from seeds of the Jojoba shrub (Simmondsia chinensis). It is made up of straight chain esters of mono-unsaturated long chain fatty acids and fatty alcohols and has an average total carbon chain length of 42 carbons, so it is classified chemically as a liquid wax. Thus, the term “jojoba oil” as used herein also includes oils substantially comprising straight chain esters of mono-unsaturated long chain fatty acids and fatty alcohols having an average total carbon chain length of 42 carbons, whether those oils are isolated from a natural source, synthetic or a combination thereof.

In one aspect, the jojoba oil used in the methods and compositions of the invention comprises a light gold fluid and has few impurities. Raw jojoba oil can have few impurities. Thus, in one aspect, the jojoba oil used in the methods and compositions of the invention comprises raw jojoba oil, requiring little or no refining. It can contain no resins, tars or alkaloids. It can contain only traces of saturated wax, steroids, tocopherols and hydrocarbons. In one aspect, the oil is not neutralized; neutralizing is usually unnecessary because the oil is normally low in free fatty acids. In one aspect, the oil is not bleached. In another aspect, the oil is processed by a commercial technique, e.g., filtration through Fuller's earth, to remove yellow pigments and produce a colorless product. The oil can be pasteurized to kill microorganisms.

In one aspect, the oil made substantially of straight chain esters of mono-unsaturated long chain fatty acids and fatty alcohols having an average total carbon chain length of about 42 carbons, e.g., jojoba oil, used in the methods and compositions of the invention is non-toxic and biodegradable. It can dissolve readily in common organic solvents such as benzene, petroleum ether, chloroform, carbon tetrachloride, and carbon disulfide. It can be immiscible with methanol and acetone.

In one aspect, the oil made substantially of straight chain esters of mono-unsaturated long chain fatty acids and fatty alcohols having an average total carbon chain length of about 42 carbons, e.g., jojoba oil, used in the methods and compositions of the invention has: low viscosity, high flash and fire points, high dielectric constant and/or high stability. In one aspect, the jojoba oil used in the methods and compositions of the invention has: low volatility and/or its composition is little affected by repeated heating to high temperatures, for example, up to 300° C.

In one aspect, the jojoba oil used in the methods and compositions of the invention has good keeping qualities and an exceptional shelf life. This may be due to the presence of natural antioxidants (alpha-, gamma- and delta-tocopherols) which occur in concentrations of about 50 ppm. These antioxidants can keep the oil from becoming rancid. In one experiment, seeds analyzed 25 years after harvest showed no change in composition. Dry seeds can be stored without deterioration or chemical changes.

In alternative aspects, the jojoba oil used in the methods and compositions of the invention are from natural or synthetic sources, or a combination thereof. The jojoba oil used in the methods and compositions of the invention can be purchased commercially as: pure, natural, golden grade; refined and bleached grade; decolorized/deodorized grade; molecular distilled grade or a combination thereof. In one aspect, the “jojoba oil” used in the methods and compositions of the invention is a synthetic or natural equivalent comprising straight chain esters of mono-unsaturated long chain fatty acids and fatty alcohols having an average total carbon chain length of about 42 carbons.

Formulation and Administration Pharmaceuticals

In one aspect, the formulations of the invention comprise pharmaceutical compositions. The formulations can comprise a pharmacologically effective amount of a composition any drug or other active agent (discussed further, below). The formulations of the invention comprising pharmaceuticals can be administered by any means. Routine means to determine drug regimens and (topical) formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa.

The formulations of the invention can include pharmaceutically acceptable carriers that can contain a physiologically acceptable compound that acts, e.g., to stabilize the composition or to increase or decrease the absorption of the pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also used to stabilize the composition or to increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound depends, e.g., on the site or route of administration and on the particular physio-chemical characteristics of any co-administered agent.

In one aspect, the formulation for administration comprises a pharmaceutically acceptable carrier, e.g., an oil made substantially of straight chain esters of mono-unsaturated long chain fatty acids and fatty alcohols having an average total carbon chain length of about 42 carbons, such as jojoba oil. A variety of additional compositions can be used in the carriers, e.g., buffered saline and the like. These formulations can be sterile and generally free of undesirable matter. These formulations may be sterilized by conventional, well-known sterilization techniques. The formulations may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The formulations of the invention can be administered in a variety of unit dosage forms, the general medical condition of each patient, the method of administration, and the like. Details on dosages are well described in the scientific and patent literature, see, e.g., the latest edition of Remington's Pharmaceutical Sciences. The exact amount of active agent in a formulation, active agent delivered, concentration of pharmaceutical in the formulations, amount of formulation in a given dose, or the “effective dose” can be routinely determined by, e.g., the clinician. The “dosing regimen,” will depend upon a variety of factors, e.g., the general state of the patient's health, age and the like. Using guidelines describing alternative dosaging regimens, the skilled artisan can determine by routine trials optimal effective concentrations of formulations of the invention. The invention is not limited by any particular dosage range.

The formulations of the invention can be delivered by any topical or mucosal means, including transmucosal delivery, e.g., buccal, bladder, vaginal, uterine, rectal, nasal mucosa. The formulations of the invention can be delivered by aerosol to have a “regional effect,” e.g., to focus on a specific organ, e.g., lungs, nasal passages, bronchi.

The pharmaceutical formulations of the invention can be presented in unit-dose or multi-dose sealed containers.

Active Compounds

The invention provides formulations comprising active agents, e.g., drugs, vitamins, and the like. Active compounds include but are not limited to pharmaceuticals, cosmetics, nutritional supplements and plant extracts. In alternative aspects, examples of active compounds comprise any antibiotics, e.g., erythromycin, tetracycline, minocycline, neomycin and mixtures thereof. In alternative aspects, examples of active compounds comprise any plant extract, e.g., aloe vera, lavender, chamomile, calendula, Echinacea, saw palmetto, green tea, gingko biloba, birch, kiwi, magnolia, peppermint, philodendron and mixtures thereof. In alternative aspects, examples of active compounds comprise any antifungal, e.g., clotrimazole, tolnaftate, terbinafine hydrochloride and mixtures thereof. In alternative aspects, examples of active compounds comprise any analgesic and/or anesthetic, e.g., benzocaine, menthol, phenol, camphor, methyl salicylate and mixtures thereof. In alternative aspects, examples of active compounds comprise any anti-aging agent, e.g., Vitamins E, A, C, B and derivatives; retinoids; antioxidants including alpha hydroxy acids such as lactic acid (e.g., DL lactic acid), glycolic acid, citric acid, malic acid, ascorbic acid and tartaric acid; beta hydroxy acids such as beta hydroxybutyric acid and beta phenyl lactic acid, DL lactic acid; plant extracts such as green tea, mulberry, genistein and daidzein from soy and mixtures thereof. In alternative aspects, examples of active compounds comprise any anti-acne agent, e.g., Benzoyl peroxide, salicylic acid, topical retinoids (retinol, tretionin, adapalene, isotretionin, azelaic acid, motretinide, tazarotene), tetracycline, erythromycin and mixtures thereof. In alternative aspects, examples of active compounds comprise any hair growth promoter, e.g., rogaine, minoxidil, Serenoa repens (saw palmetto) extract, finasteride, PROPECIA™ and mixtures thereof. In alternative aspects, examples of active compounds comprise any anti-dandruff agent, anti-psoriasis agent, anti-seborrheic agent, anti-dermatitis agent, e.g., coal tar, selenium sulfide, sulfur, zinc pyrithione, salicylic acid, ketoconazole, clotrimazole, miconazole, fluconazole, vitamin A analogs, corticosteroids and mixtures thereof. In alternative aspects, examples of active compounds comprise any vitamin, e.g., vitamin B complex; including thiamine, biotin, riboflavin, vitamin B6, vitamin B12; vitamins A, C, D, E, K, their derivatives and mixtures thereof. In alternative aspects, examples of active compounds comprise any amino acid, e.g., cysteine, glutamic acid, glycine, alanine, serine, valine, methionine, tryptophan, leucine and mixtures thereof.

In one aspect, the formulation comprises a lactic acid, e.g., DL-lactic acid, an exemplary formulation is discussed in Example 6, below.

Topical Formulations

The invention provides topical formulations comprising an active agent and an oil made substantially of straight chain esters of mono-unsaturated long chain fatty acids and fatty alcohols having an average total carbon chain length of about 42 carbons, e.g., jojoba oil. Topical formulations may be in any form suitable for application to any body surface. Topical formulations may comprise an aqueous solution, an ointment, a cream, a gel, a lotion, a paste and the like. Topical formulations may be prepared so as to contain liposomes, micelles, and/or microspheres. Topical formulations can be sprays, sticks, ointments, creams or a foam.

In one aspect, the formulation comprises an ointment, e.g., a semisolid preparation comprising petrolatum or other petroleum derivatives. In one aspect, the specific ointment base provides for desired characteristics, e.g., emolliency. As with other carriers or vehicles, an ointment base can be inert, stable, nonirritating and nonsensitizing. In alternative aspects, ointment bases are oleaginous bases; emulsifiable bases; emulsion bases; and water-soluble bases (see, e.g., Remington). In alternative aspects, oleaginous ointment bases comprise vegetable oils, fats obtained from animals, and semisolid hydrocarbons obtained from petroleum. In alternative aspects, emulsifiable ointment bases, also known as absorbent ointment bases, contain little or no water and include, for example, hydroxystearin sulfate, anhydrous lanolin and hydrophilic petrolatum. In alternative aspects, emulsion ointment bases are either water-in-oil (W/O) emulsions or oil-in-water (O/W) emulsions, and include, for example, cetyl alcohol, glyceryl monostearate, lanolin and stearic acid. Water-soluble ointment bases can be prepared from polyethylene glycols of varying molecular weight; see, e.g., Remington.

In alternative aspects, the formulation comprises creams, e.g., viscous liquids or semisolid emulsions, either oil-in-water or water-in-oil. Cream bases can be water-washable, and can contain an oil phase, an emulsifier and an aqueous phase. The oil phase can be petrolatum or a fatty alcohol such as cetyl or stearyl alcohol. The aqueous phase can exceed the oil phase in volume and can contain a humectant. The emulsifier in a cream formulation can comprise a nonionic, anionic, cationic or amphoteric surfactant.

In alternative aspects, the formulation comprises gels, such as semisolid or suspension-type systems. In one aspect, the formulation comprises single-phase gels containing organic macromolecules distributed substantially uniformly throughout the carrier liquid which can also comprise an alcohol and/or an oil.

In alternative aspects, the formulation comprises “organic macromolecules,” i.e., gelling agents, that can be crosslinked acrylic acid polymers, such as the “carbomer” family of polymers, e.g., carboxypolyalkylenes.

In alternative aspects, the formulation comprises hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin.

In one aspect, in order to prepare a uniform gel, dispersing agents such as alcohol or glycerin can be added, or the gelling agent can be dispersed by trituration, mechanical mixing, and/or stirring. In alternative aspects, the formulation comprises various additives. For example, solubilizers (in addition to oil, e.g., jojoba oil) may be used, e.g., to solubilize certain active agents.

### EXAMPLES

The following examples are offered to illustrate, but not to limit the claimed invention.

### Example 1

### Delivery of an Active Agent Into Hair Follicles and Sebaceous Glands

The following example describes an exemplary method of the invention and demonstrates that the compositions and methods of the invention are effective for delivery of active agents into hair follicles and sebaceous glands. The following describes the comparative testing of exemplary compositions and methods of the invention to deliver an active agent, in this example, a fluorescent dye, into hair follicles and sebaceous glands.

Mixtures of Nile Red fluorescent dye, were from Sigma Chemical Co., St. Louis, Mo., with delivery agents were prepared as follows:
A. 5 mg of Nile Red were added to 4 gram (gr) of Jojoba Oil and mixed in a magnetic stirrer for 12 hours.
B. 5 mg of Nile Red were added to 4 gr of Diethylene Glycol Monoethyl Ether (Transcutol, Lipscomb Chemical Company, Inc., Long Beach, Calif.) and mixed in a magnetic stirrer for 12 hours.
C. 5 mg of Nile Red were added to 4 gr of Dimethyl Sulfoxide (DMSO) and mixed in a magnetic stirrer for 12 hours.
D. 5 mg of Nile Red were added to a mixture of 1.2 gr Ethanol, 2 gr Propylene Glycol and 0.8 gr of distilled water (this mixture is similar in composition to the vehicle of rogaine) and mixed in a magnetic stirrer for 12 hours.

All mixtures were filtered through a 0.2 micron Millipore filter, available from Millipore Corp., Bedford, Mass., to remove any undissolved Nile Red dye.

200 microliters of the Nile Red-containing compositions A, B, C and D, respectively, were applied via pipette to approximately 2.5 square cm of the dorsal skin of 4 Sprague Dawley rats, available from Charles River Laboratory, Kingston, N.Y., twice daily, morning and afternoon, for 5 days. Before the application of formulations, the hair of the rats was gently clipped with an electric clipper on their dorsal areas. For negative control purposes, one similar Sprague Dawley rat was left untreated after its hair was clipped.

Samples of the treated skin from each rat, including the negative control, were removed after 5 days, embedded in O.C.T compound, available from Sakura Finetek USA, Inc., Torrance, Calif. and frozen in dry ice-cooled 2-methylbutane, available from Sigma Aldrich, Milwaukee, Wis. 50-micron-thick frozen sections were cut from the O.C.T embedded skin samples in a cryostat, available from Leica, Inc., Deerfield, Ill. and observed under a fluorescent microscope equipped with a 460 nm filter, available from Olympus America, Inc., Melville, N.Y.

Representative digital photomicrographs, light and fluorescent microscopy, of hair follicles and sebaceous glands from skin frozen sections of each rat were taken using a Nikon Coolpix 995 digital camera, available form Nikon, Inc., Melville, N.Y.

The photomicrographs were observed to determine the extent of delivery of Nile Red dye into the hair follicles and sebaceous glands of the dorsal skin of each treated animal. The amount of Nile Red delivered is evidenced by the intensity of the orange-red fluorescence depicted in the photomicrographs. Green auto-fluorescence from the hair shafts, sebaceous glands and to a lesser extent from the surrounding dermis is not due to fluorescence from Nile Red and was not evaluated. Orange-red fluorescence due to Nile Red dye on the skin surface (stratum corneum) was not evaluated.

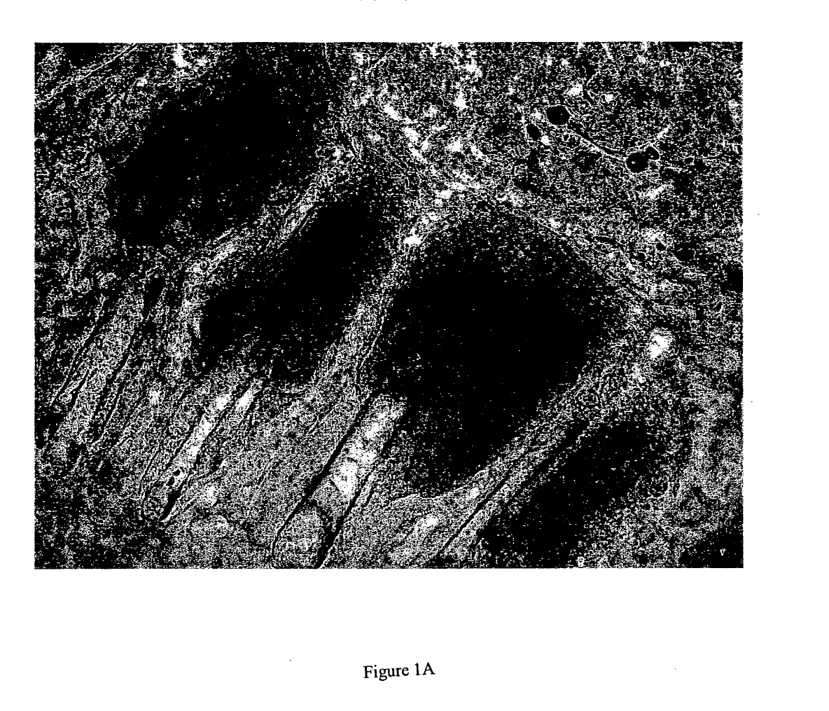

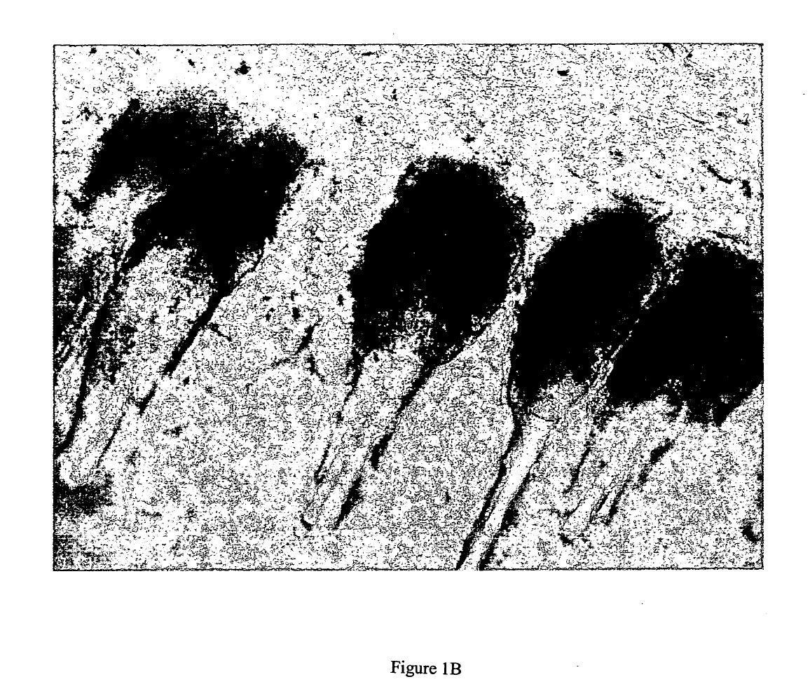

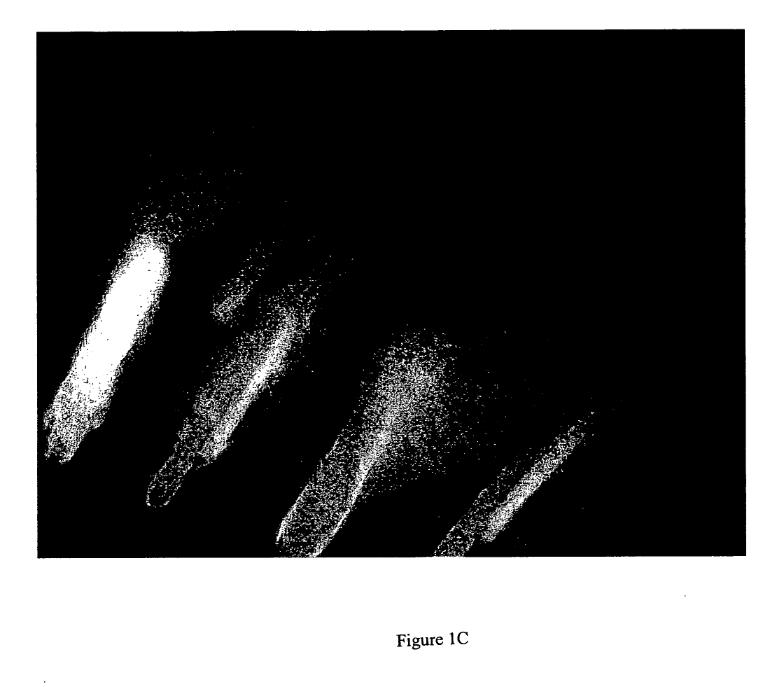

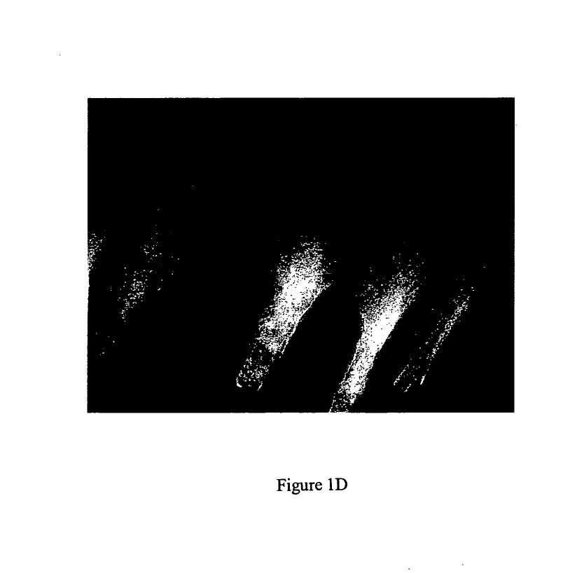

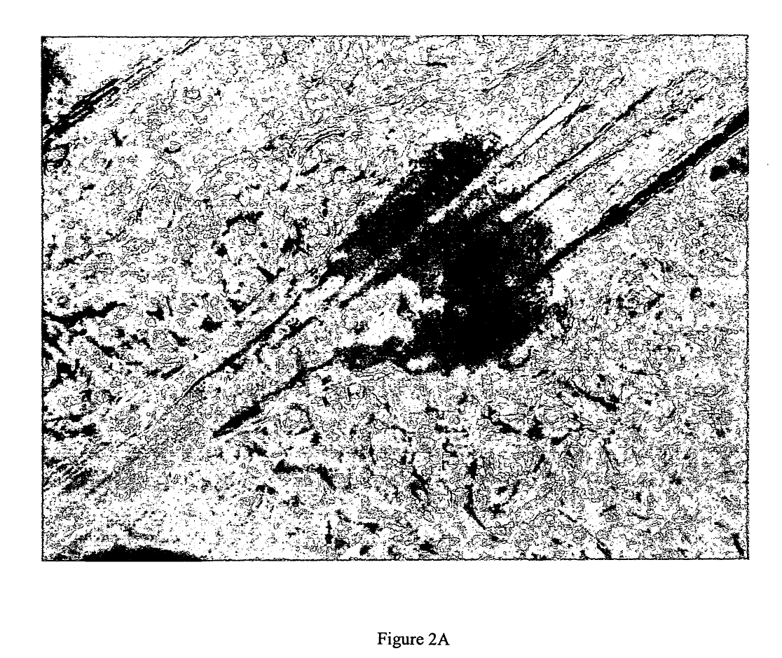

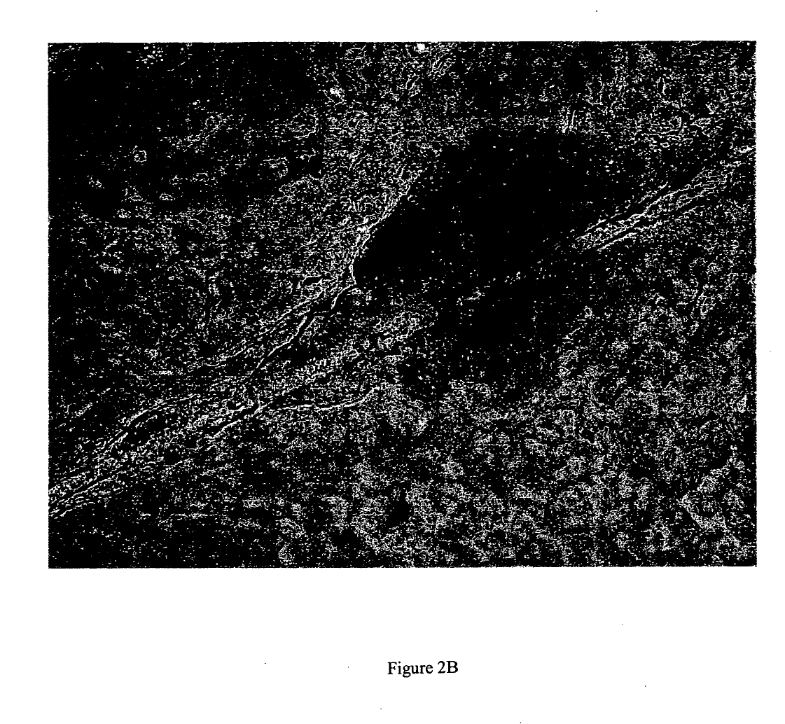

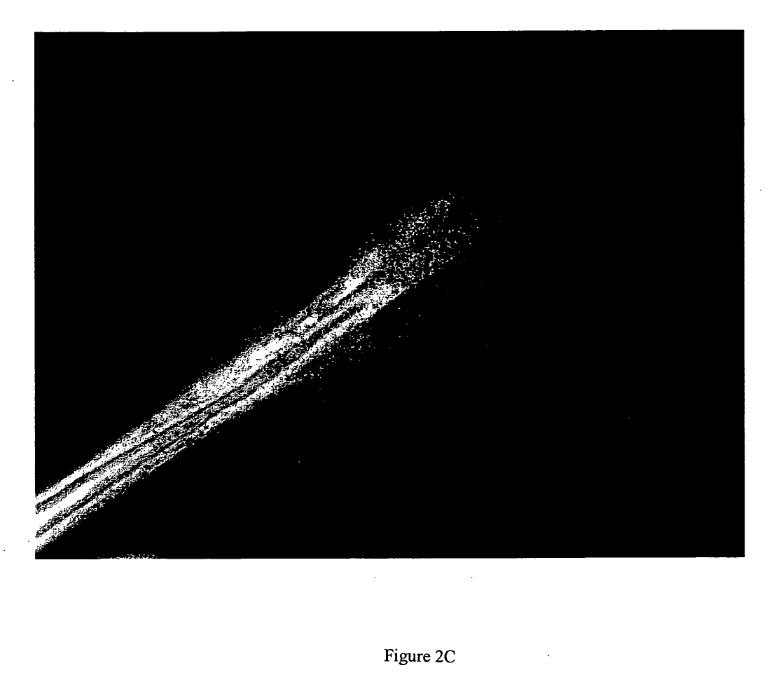

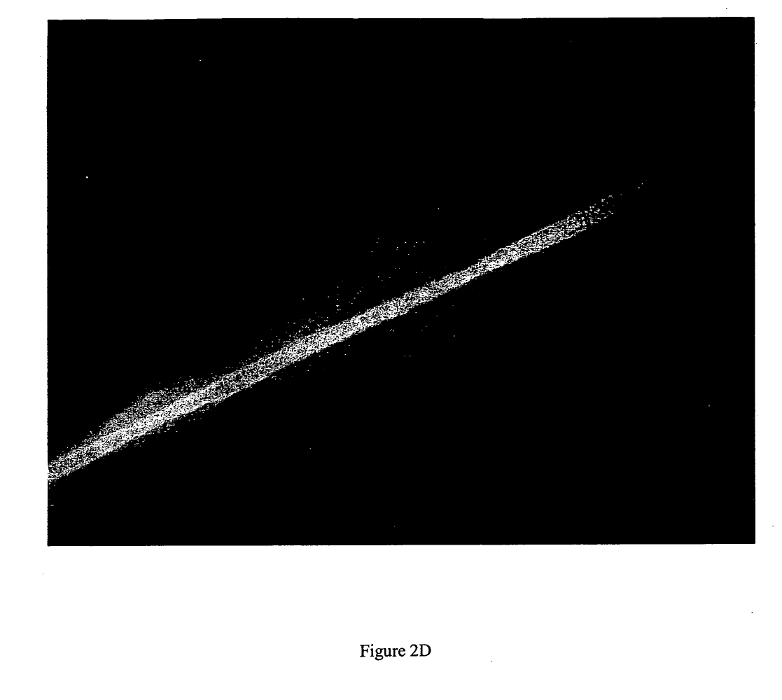

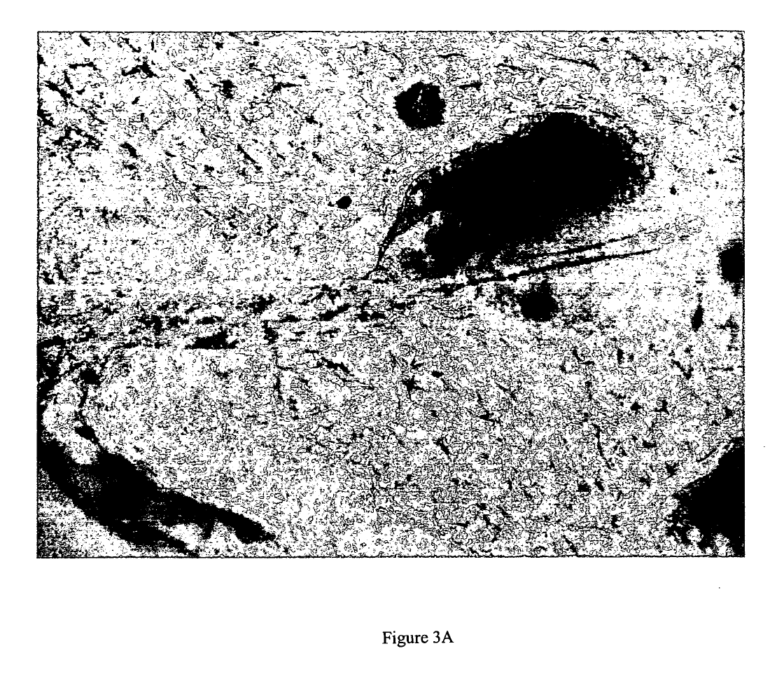

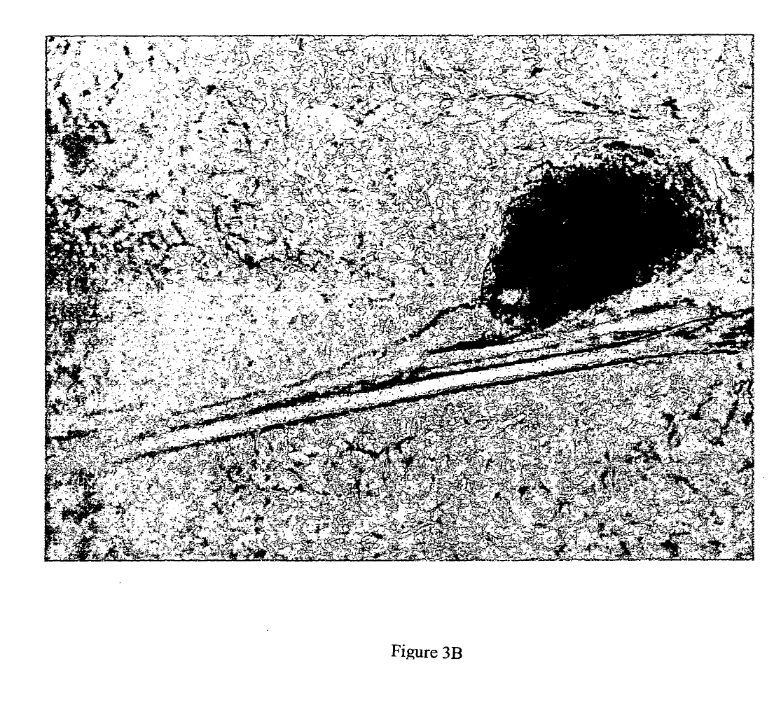

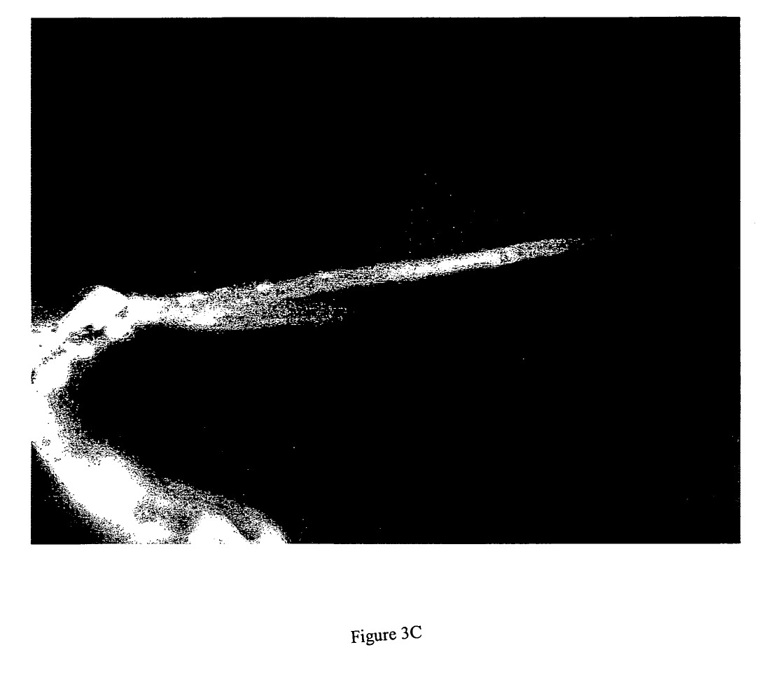

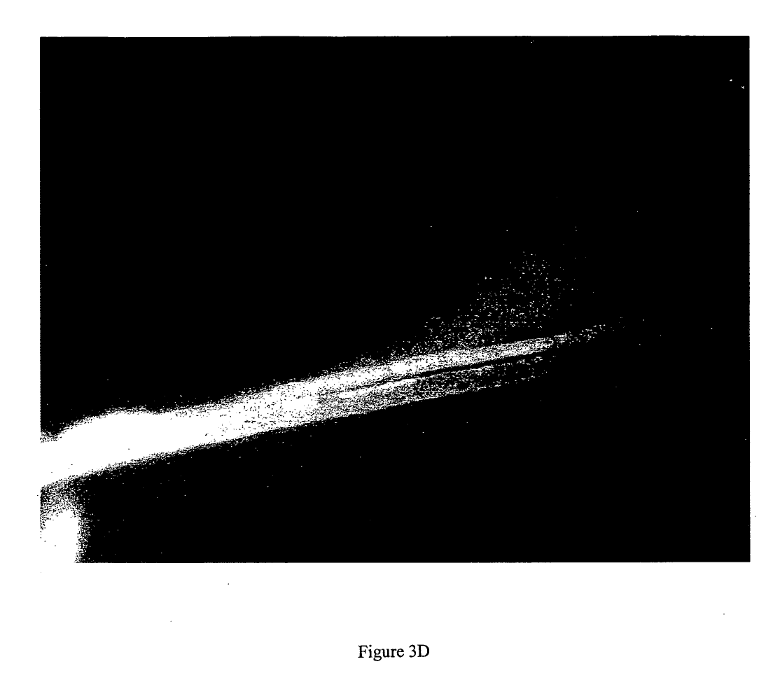

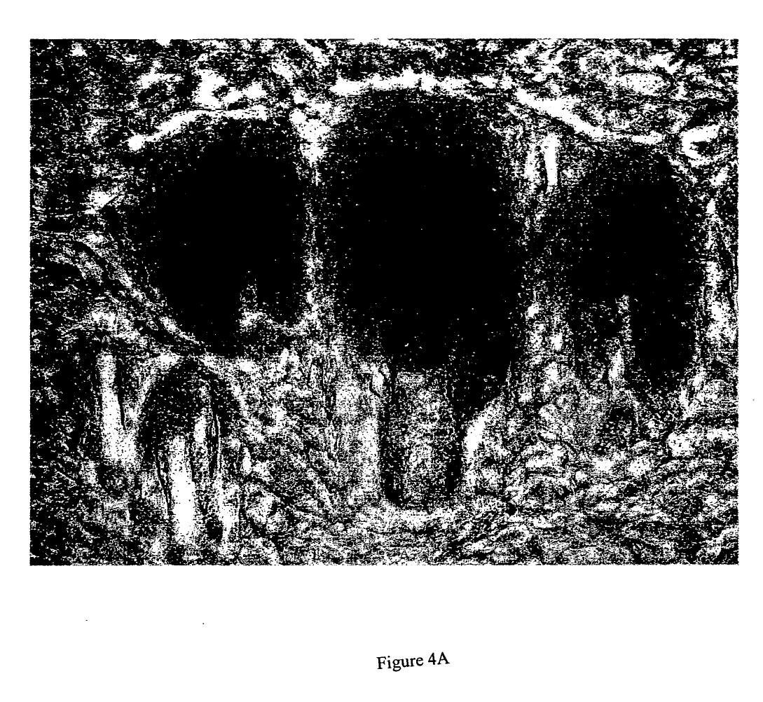

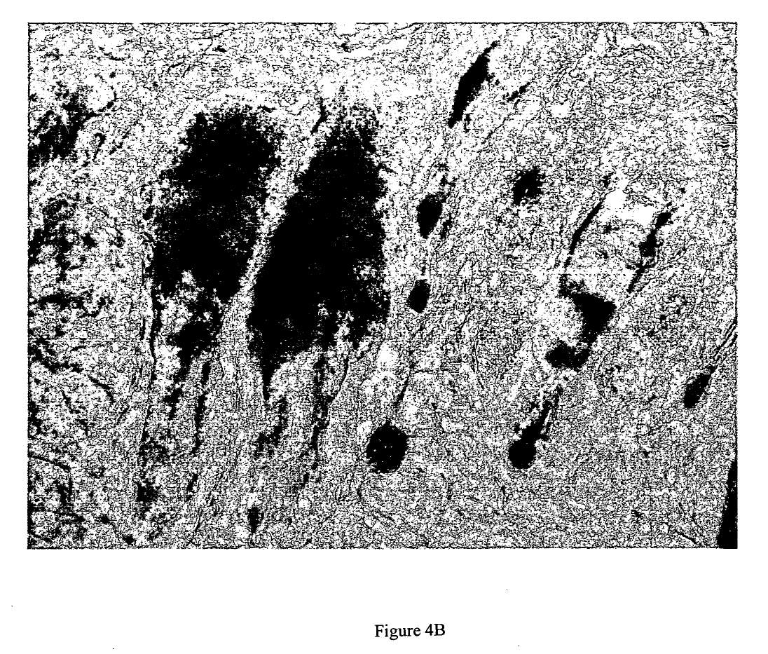

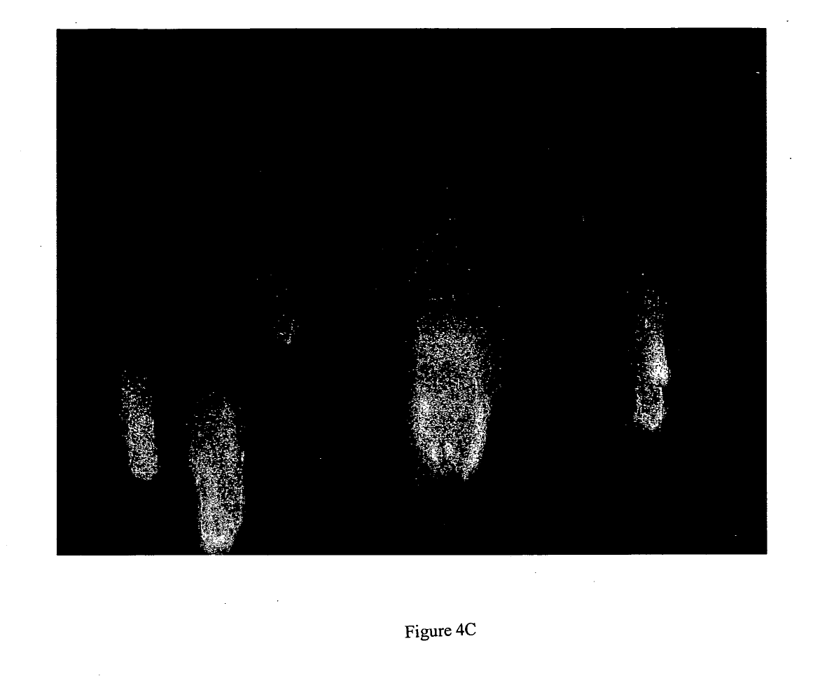

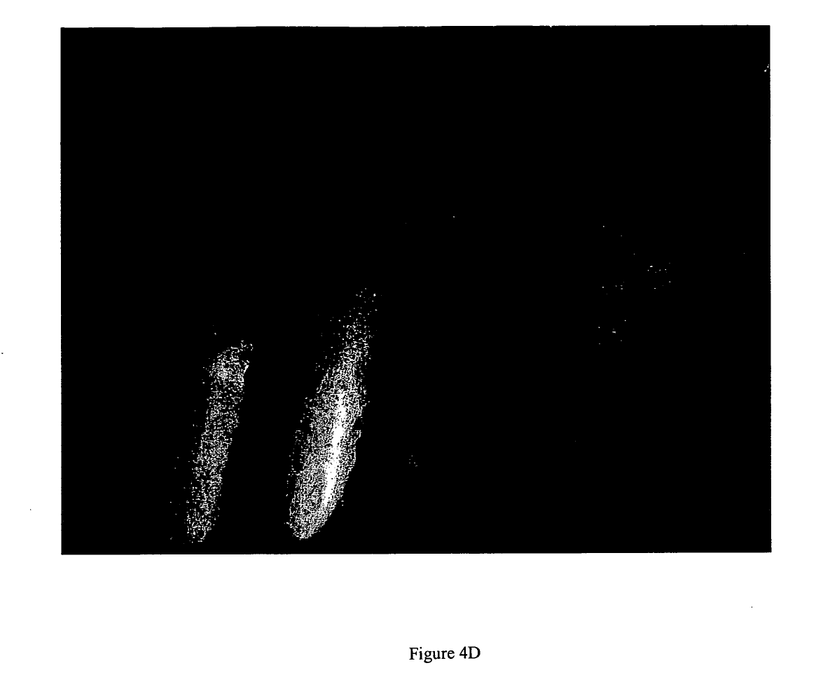

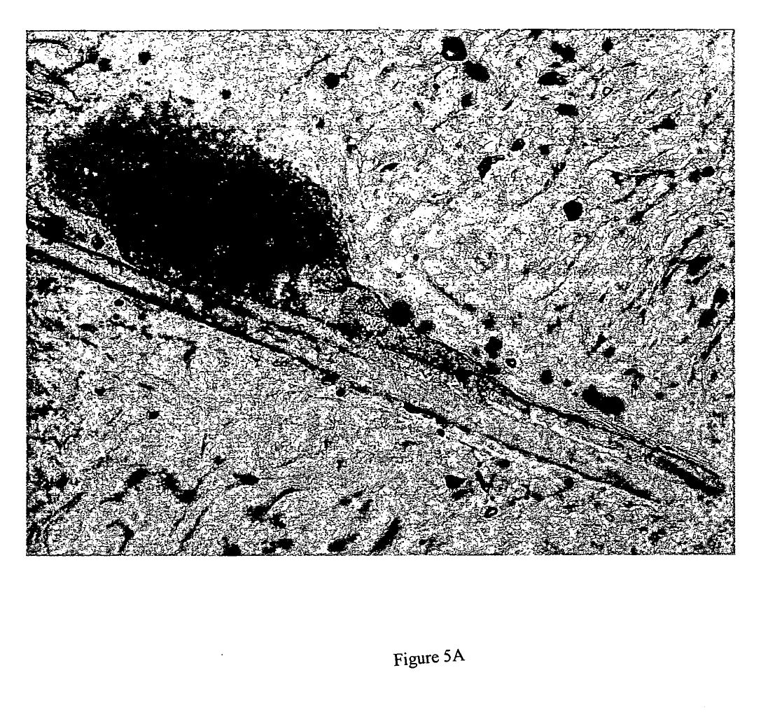

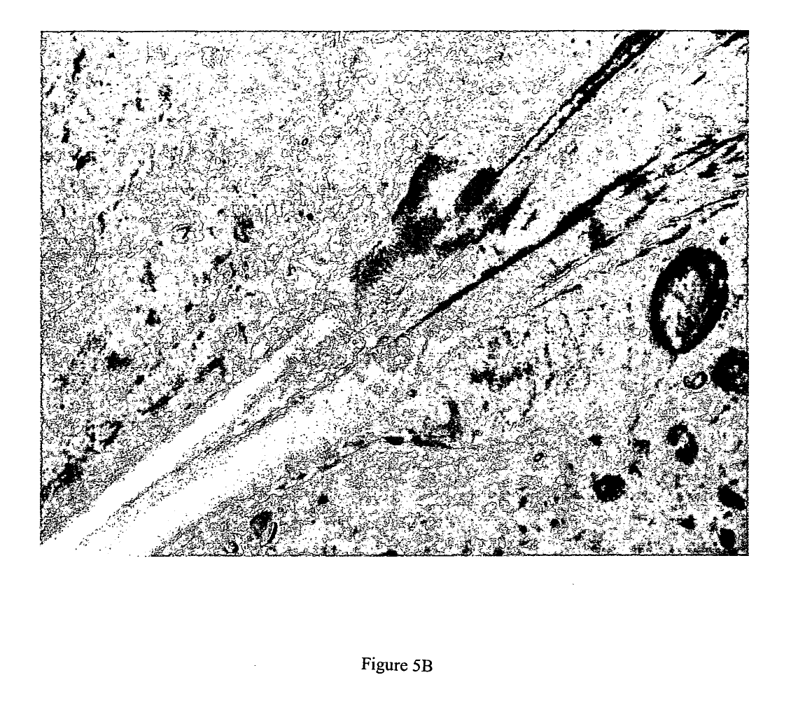

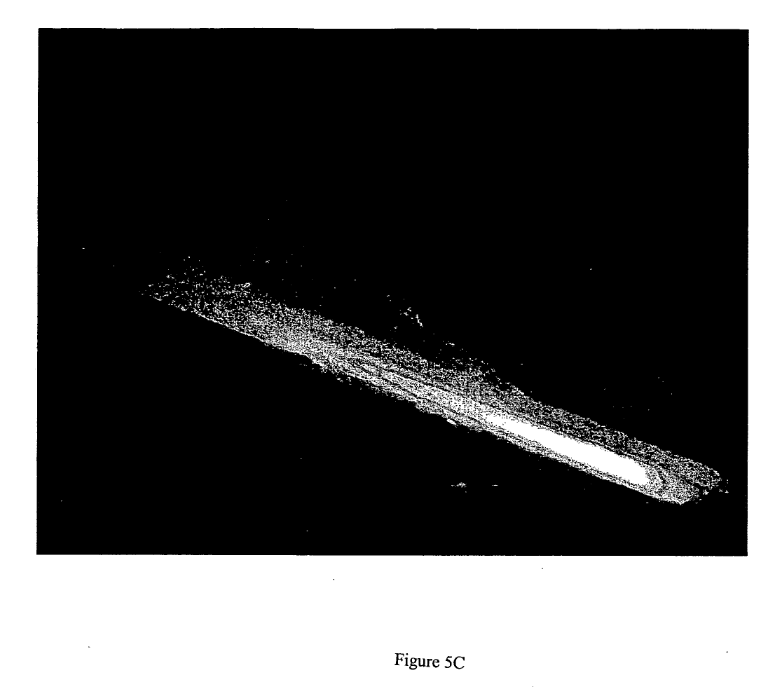

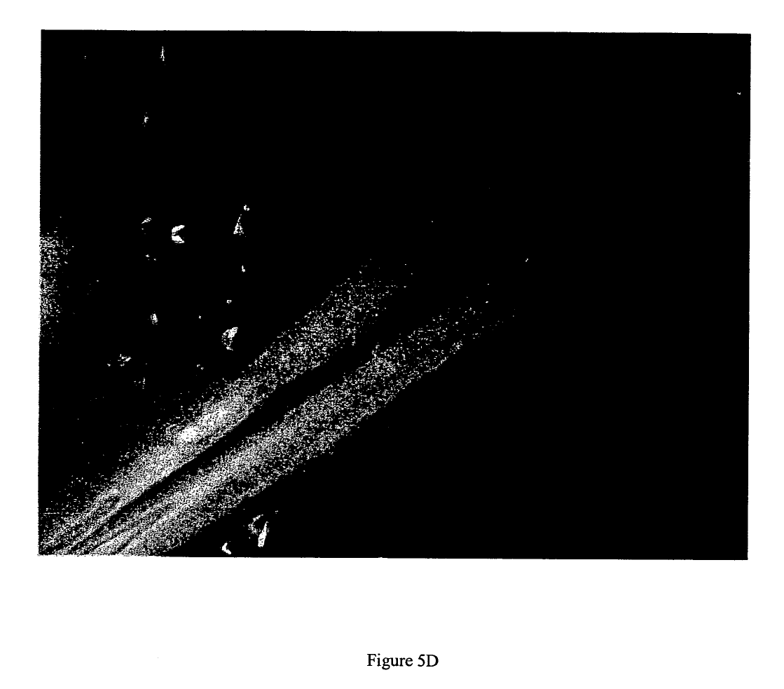

FIG. 1A and FIG. 1B illustrate histological views of the dorsal skin of Sprague Dawley rat treated with a solution of 0.125% Nile Red in Jojoba Oil in unstained frozen sections, 50 micron thick, Light microscopy. FIG. 1C and FIG. 1D illustrate the corresponding histological views to FIGS. 1A and 1B observed under fluorescence microscopy. FIG. 2A and FIG. 2B illustrate histological views of the dorsal skin of Sprague Dawley rat treated with a solution of 0.125% Nile Red in Diethylene Glycol Monoethyl Ether (Transcutol) in unstained frozen sections, 50 micron thick, Light microscopy. FIG. 2C and FIG. 2D illustrate corresponding histological views to FIGS. 2A and 2B observed under fluorescence microscopy. FIG. 3A and FIG. 3B illustrate histological views of the dorsal skin of Sprague Dawley rat treated with a solution of 0.125% Nile Red in Dimethyl Sulfoxide (DMSO) in unstained frozen sections, 50 micron thick, Light microscopy. FIGS. 3C and 3D illustrate corresponding histological views to FIGS. 3A and 3B observed under fluorescence microscopy. FIG. 4A and FIG. 4B illustrate histological views of the dorsal skin of Sprague Dawley rat treated with a solution of 0.125% Nile Red in a solution containing 50% Propylene glycol, 30% Ethanol and 20% Water, unstained frozen sections, 50 micron thick, Light microscopy. FIG. 4C and FIG. 4D illustrate corresponding histological views to FIGS. 4A and 4B observed under fluorescence microscopy. FIG. 5A and FIG. 5B illustrates histological views of untreated dorsal skin of Sprague Dawley rat in unstained frozen sections, 50 micron thick, Light microscopy. FIG. 5C and FIG. 5D illustrate corresponding histological views to FIGS. 5A and 5B observed under fluorescence microscopy.

| Vehicle | Fluorescence intensity |
|---|---|
| Jojoba Oil | ++++ |
| DMSO | +++ |
| Transcutol | ++ |
| Rogaine vehicle | + |
| Untreated control | 0 |

### Example 2

### An Exemplary Formulation of the Invention for the Treatment of Acne

The following example describes an exemplary formulation of the invention that can be used, e.g., for the treatment of acne. In alternative aspects, the formulation comprises, based on the total weight of the composition, from about 0.005 percent to about 5 percent, or, about 0.01 percent to about 3 percent, or, from about 0.1 percent to 2.5 percent, or, from about 1 percent to 2 percent of benzoyl peroxide.

Preparation of Jojoba Oil with Benzoyl Peroxide: 2.0 g of Benzoyl Peroxide, available from Sigma Chemical Co., St. Louis, Mo., were added while stirring at room temperature (RT) into a beaker containing 98.0 g of Jojoba Oil. The temperature of the mixture was raised to 40° C. and maintained with constant stirring until complete dissolution of the Benzoyl Peroxide. The mixture was allowed to cool to room temperature without stirring and packaged in 30 ml plastic droppers.

The composition is applied to the affected area. In one aspect, it is applied at night before going to sleep. In one aspect, the affected area is not wetted or washed before application because water may diminish the ability of the oil to penetrate the skin. A few drops of the mixture can be applied to provide a thin coat and gently rubbed with the fingertips. It can be left overnight to allow the oil with Benzoyl Peroxide to penetrate into the skin, e.g., the sebaceous glands. The treated area can be washed the next morning with a mild soap to remove any remaining composition on the surface of the skin.

This composition can help prevent acne and minimize outbreaks. The drying and sometimes irritating effect of the Benzoyl Peroxide can be substantially eliminated or minimized by the emollient effect of the Jojoba Oil. In one aspect, the low concentration of Benzoyl Peroxide and beneficial effects of the oil allow this composition to be used daily without detriment to the skin.

### Example 3

### An Exemplary Formulation of the Invention for the Treatment of Hair Loss

The following example describes an exemplary formulation of the invention for the treatment of, e.g., hair loss. In alternative aspects, the formulation comprises, based on the total weight of the composition, from about 0.005 percent to about 5 percent, or, about 0.01 percent to about 3 percent, or, from about 0.1 percent to 2.5 percent, or, from about 1 percent to 2 percent of minoxidil.

Preparation of Jojoba Oil with minoxidil: Powdered minoxidil, available from Spectrum Chemicals and Laboratory Products, Inc., Gardena, Calif. is manually ground on a glass mortar to reduce the size of the largest crystals to below 100 microns. One gram of the ground minoxidil is added with stirring to 99 grams of Jojoba Oil. The mixture is homogenized at high pressure (18,000 psi) utilizing the M-110F. Microfluidizer, available from Microfluidics Corp., Newton, Mass. In alternative aspect the mixing is done at 10,000 rpm utilizing the L4RT High Shear Mixer, available from Silverson Machines, Inc., East Longmeadow, Mass. This process creates an ultra-fine suspension of minoxidil in Jojoba Oil with sub-micron particle sizes, small enough to penetrate the hair follicle and the sebaceous gland.

The composition can be applied to the scalp, e.g., at night before going to sleep. In one aspect, the scalp is not wetted or washed before application because water may diminish the ability of the oil to penetrate the skin. In one aspect, a few drops of the mixture are applied to the affected area of the scalp to provide a thin coat and gently rubbed with the fingertips. The formulation can be left overnight to allow the oil with the minoxidil to penetrate into the skin, e.g., the hair follicle and sebaceous glands. A morning shower with a gentle shampoo will remove any residual composition from the scalp. Besides being an ideal delivery agent for minoxidil, Jojoba Oil alone can promote healthy hair. This formulation is gentle enough to be used daily without adverse effects to the scalp.

### Example 4

### An Exemplary Formulation of the Invention for the Treatment of Hair Loss

The following example describes an exemplary formulation of the invention for the treatment of, e.g., hair loss. In alternative aspects, the formulation comprises, based on the total weight of the composition, from about 0.005 percent to about 5 percent, or, about 0.01 percent to about 3 percent, or, from about 0.1 percent to 2.5 percent, or, from about 1 percent to 2 percent of finasteride, or, a mixture comprising finasteride and minoxidil. In one aspect, the mixture comprises from about 0.005 percent to about 5 percent, or, about 0.01 percent to about 3 percent, or, from about 0.1 percent to 2.5 percent, or, from about 1 percent to 2 percent of finasteride and from about 0.005 percent to about 5 percent, or, about 0.01 percent to about 3 percent, or, from about 0.1 percent to 2.5 percent, or, from about 1 percent to 2 percent of minoxidil.

Preparation of Jojoba Oil with minoxidil and finasteride: Powdered minoxidil, available from Spectrum Chemicals and Laboratory Products, Inc., Gardena, Calif. is manually ground on a glass mortar to reduce the size of the largest crystals to below 100 microns. One gram of the ground minoxidil is added with stirring to 98.5 grams of Jojoba Oil.

Powdered finasteride, available from Sigma Chemical Company, St. Louis, Mo., is ground (e.g., manually ground) on a glass mortar to reduce the size of the largest crystals to below 100 microns. Half of one gram of the ground finasteride is added with stirring to the previous mixture.

The mixture is homogenized at high pressure (18,000 psi) utilizing the M-110F Microfluidizer, available from Microfluidics Corp., Newton, Mass. In alternative aspect the mixing is done at 10,000 rpm utilizing the L4RT High Shear Mixer, available from Silverson Machines, Inc., East Longmeadow, Mass. This process creates an ultra-fine suspension of minoxidil and finasteride in Jojoba Oil with sub-micron particle sizes, small enough to penetrate the hair follicles and the sebaceous glands.

In one aspect, the composition is applied to the scalp, e.g., at night before going to sleep. In one aspect, the scalp is not wetted or washed before application because water may diminish the ability of the oil to penetrate the skin. In one aspect, a few drops of the mixture are applied to the affected area of the scalp to provide a thin coat and gently rubbed with the fingertips. In one aspect, the formulation is left overnight to allow the oil with minoxidil and finasteride to penetrate into the skin, e.g., the hair follicles and sebaceous glands. A morning shower with a gentle shampoo may remove any residual composition from the scalp. Besides being an ideal delivery agent for minoxidil and finasteride, Jojoba Oil alone can promote healthy hair. This formulation is gentle enough to be used daily without adverse effects to the scalp.

### Example 5

### An Exemplary Formulation of the Invention for the Treatment of Hair Loss or Promotion of Hair Growth

The following example describes an exemplary formulation of the invention for the treatment of, e.g., hair loss or promotion of hair growth. In alternative aspects, the formulation comprises, based on the total weight of the composition, from about 0.005 percent to about 5 percent, or, about 0.01 percent to about 3 percent, or, from about 0.1 percent to 2.5 percent, or, from about 1 percent to 2 percent minoxidil and Serenoa repens (saw palmetto) extract, e.g., from about 0.005 percent to about 5 percent, or, about 0.01 percent to about 3 percent, or, from about 0.1 percent to 2.5 percent, or, from about 1 percent to 2 percent of Serenoa repens (saw palmetto) extract.

Preparation of Jojoba Oil with minoxidil and Serenoa repens (saw palmetto) extract: Powdered minoxidil, available from Spectrum Chemicals and Laboratory Products, Inc., Gardena, Calif. is manually ground on a glass mortar to reduce the size of the largest crystals to below 100 microns. One gram of the ground minoxidil is added with stirring to 98.0 grams of Jojoba Oil. One gram of Serenoa repens (saw palmetto) extract 85-95%, available from The Saw Palmetto Harvesting Company, Frostproof, Fla. is added with stirring to the previous mixture.

The mixture is homogenized at high pressure (18,000 psi) utilizing the M-110F Microfluidizer, available from Microfluidics Corp., Newton, Mass. In alternative aspect the mixing is done at 10,000 rpm utilizing the L4RT High Shear Mixer, available from Silverson Machines, Inc., East Longmeadow, Mass. This process creates an ultra-fine suspension of minoxidil in Jojoba Oil with sub-micron particle sizes, small enough to penetrate the hair follicles and the sebaceous glands. The saw palmetto extract is completely miscible in Jojoba Oil.

In one aspect, the composition is applied to the scalp at night before going to sleep. In one aspect, the scalp is not wetted or washed before application because water may diminish the ability of the oil to penetrate the skin. In one aspect, a few drops of the mixture are applied to the affected area of the scalp to provide a thin coat and gently rubbed with the fingertips. In one aspect, the formulation is left overnight to allow the oil with minoxidil and Serenoa repens (saw palmetto) extract penetrate into the skin, e.g., the hair follicles and sebaceous glands. A morning shower with a gentle shampoo may remove any residual composition from the scalp. Besides being an ideal delivery agent for minoxidil and Serenoa repens (saw palmetto) extract, Jojoba Oil alone may promote healthy hair. This formulation is gentle enough to be used daily without adverse effects to the scalp.

### Example 6

### An Exemplary Formulation of the Invention

The following example describes an exemplary formulation of the invention comprising an emulsion comprising DL lactic acid. In one aspect, the emulsion is made with an insoluble liquid (not a solid) compound in Jojoba Oil; emulsions of this type will be “water in oil”.

In one aspect, a micro-emulsion of the liquid active compound in Jojoba Oil is prepared by using a high pressure Microfluidizer-homogenizer. Process pressures range from 3,000 to 23,000 psi. In alternative aspect, a micro emulsion of the liquid active compound in Jojoba Oil is prepared by using a rotor-stator High Shear Mixer-homogenizer. Mixing speeds range from 1,000 rpm to 25,000 rpm. Insoluble liquid compounds are mixed with Jojoba Oil, accelerated to high velocities and subjected to high shear rates to produce uniform sub-micron droplets.

In one aspect, with respect to treatments for anti-aging, the composition of the present invention comprises, based on the total weight of the composition, from about 0.01 percent to about 5 percent, or, from about 0.1 percent to 4 percent, or, from about 1 percent to 4 percent, or, from about 2 percent to 4 percent of DL-lactic acid.

Preparation of Jojoba Oil with DL-lactic acid: 3 grams of Liquid DL-Lactic Acid, available from Sigma Chemical Co. St. Louis, Mo. are added with stirring to 97 grams of Jojoba Oil. The mixture is homogenized at high pressure (3,000 to 23,000 psi) utilizing the M-110F Microfluidizer, available from Microfluidics Corp., Newton, Mass. This process creates a micro-emulsion of DL-lactic acid in Jojoba Oil with sub-micron particle droplets.

In one aspect, the composition is applied to the face, e.g., at night before going to sleep. In one aspect, the face is not wetted or washed before application because water may diminish the ability of the oil with DL-lactic acid to penetrate the skin. In one aspect, a few drops of the mixture are applied to the skin of the face to provide a thin coat and gently rubbed with the fingertips. The formulation can be left overnight to allow the oil with DL-lactic acid to act on the skin, e.g., the stratum corneum. Washing the face with a gentle soap may remove any residual composition from the skin.

A number of embodiments of the invention have been described. Nevertheless, it will be understood that various modifications may be made without departing from the spirit and scope of the invention. Accordingly, other embodiments are within the scope of the following claims.

1. A topical formulation comprising an active compound and jojoba oil, wherein the formulation is made by a method comprising the following steps: (a) providing an active compound and a jojoba oil; and (b) mixing the active compound and the jojoba oil at room temperature without the use of heat until complete dissolution of the compound.

2. The topical formulation of claim 1, wherein room temperature is between about 20° C. to 25° C.

3. A topical formulation comprising an active compound and jojoba oil, wherein the formulation is made by a method comprising the following steps: (a) providing an active compound and a jojoba oil; and (b) mixing the active compound and the jojoba oil with heat until complete dissolution of the compound.

4. The topical formulation of claim 3, wherein the active compound and the jojoba oil are mixed at temperatures above about 25° C., or above about 35° C., 45° C., 55° C., 65° C., 75° C., 85° C. or 95° C.

5-6. (canceled)

7. A topical formulation comprising an active compound and jojoba oil, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution,
wherein optionally the solubility enhancing agent is selected from the group consisting of an ethanol, a polyol or a mixture thereof,
and optionally the polyol is selected from the group consisting of propylene glycol, glycerol and polyethylene glycol.

8-10. (canceled)

11. A topical formulation comprising a fine particle dispersion of an active compound in jojoba oil, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil,
wherein optionally the mixture is accelerated to pressures ranging from about 3,000 to about 23,000 psi,
and optionally the mixing is done in amicrofluidizer-homogenizer or in a rotor-stator High Shear Mixer-homogenizer, and optionally the high shear rates produce micron-sized particles,
and optionally the formulation comprises between about 5% and 99% jojoba oil.

12-51. (canceled)

52. The topical formulation of claim 1, wherein the active compound comprises a hair growth promoter,
wherein optionally the hair growth promoter comprises rogaine, minoxidil, Serenoa repens (saw palmetto) extract, finasteride, PROPECIA™, or a mixture thereof.

53-59. (canceled)

60. An article of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; and (b) mixing the active compound and the jojoba oil at room temperature without the use of heat until complete dissolution of the compound.

61. (canceled)

62. An article of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; and (b) mixing the active compound and the jojoba oil with heat until complete dissolution of the compound.

63-65. (canceled)

66. An article of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solutions
wherein optionally the solubility enhancing agent is selected from the group consisting of an ethanol, a polyol or a mixture thereof, and optionally the polyol is selected from the group consisting of propylene glycol, glycerol and polyethylene glycol.

67-69. (canceled)

70. An article of manufacture comprising a packaging material and a formulation contained within the packaging material, where the formulation comprises an active compound and jojoba oil, and the packaging material comprises a label which indicates the formulation may be administered for delivering the active compound to the hair follicles or sebaceous glands or to the skin by topical application, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oils,
wherein optionally the formulation comprises an emulsion, a paste, a gel, a cream, a lotion, an aqueous solution, a foam or an ointment, or a spray or a stick.

71-114. (canceled)

115. A method for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil by stirring at room temperature without the use of heat until complete dissolution of the compound.

116. (canceled)

117. A method for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil with heat until complete dissolution of the compound.

118-120. (canceled)

121. A method for delivering an active compound into a hair follicle, a sebaceous gland or into skin comprising topical application to a subject of a formulation comprising an active compound and a jojoba oil, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution.

122-124. (canceled)

125. A method for delivering an active compound into a hair follicle, a sebaceous gland or into skin comprising topical application to a subject of a formulation comprising an active compound and a jojoba oil, wherein the formulation is made by a method comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil.

126-130. (canceled)

131. A method for delivering a compound into the stratum corneum comprising topical application to a subject a formulation comprising the compound and a jojoba oil, wherein the formulation is made by a method comprising the following steps: (a) providing an active compound and a jojoba oil; (b) mixing the active compound and the jojoba oil by stirring at room temperature without the use of heat until complete dissolution of the compound.

132. (canceled)

133. A method for delivering a compound into the stratum corneum comprising topical application to a subject a formulation comprising the compound and a jojoba oil, wherein the formulation is made by a method comprising the following steps: (a) providing an active compound and a jojoba oil; (b) mixing the active compound and the jojoba oil with heat until complete dissolution of the compound.

134 to 136 (canceled)

137. A method for delivering a compound into the stratum corneum comprising topical application to a subject of a formulation comprising the compound and a jojoba oil, wherein the formulation is made by a method comprising the following steps: (a) providing an active compound, a solubility enhancing agent and a jojoba oil; (b) dissolving the active compound into a solution comprising a solubility enhancing agent; and (c) mixing the solution of step (b) into the jojoba oil until complete dissolution of the compound into the solution.

138-146. (canceled)

147. A topical formulation comprising a hair growth promoter and jojoba oil, wherein the formulation is made by the method of claim 1, and the formulation comprises, based on the total weight of the formulation, from about 0.005 percent to about 5 percent, or, about 0.01 percent to about 3 percent, or, from about 0.1 percent to 2.5 percent, or, from about 1 percent to 2 percent of hair growth promoter.

148. (canceled)

149. A topical formulation comprising an acne ameliorating agent and jojoba oil, wherein the formulation is made by the method of claim 1, and the formulation comprises, based on the total weight of the formulation, from about 0.005 percent to about 5 percent, or, about 0.01 percent to about 3 percent, or, from about 0.1 percent to 2.5 percent, or, from about 1 percent to 2 percent of acne ameliorating agent,
wherein optionally the acne ameliorating agent comprises benzoyl peroxide.

150. (canceled)

151. A topical formulation comprising an active compound and an oil, wherein the formulation is made by a method comprising the following steps: (a) providing an active compound and an oil made substantially of straight chain esters of mono-unsaturated long chain fatty acids and fatty alcohols having an average total carbon chain length of about 42 carbons; and (b) mixing the active compound and the oil at room temperature without the use of heat until complete dissolution of the compound.

152. (canceled)

153. A topical formulation comprising an active compound and an oil, wherein the formulation is made by a method comprising the following steps: (a) providing an active compound and an oil made substantially of straight chain esters of mono-unsaturated long chain fatty acids and fatty alcohols having an average total carbon chain length of about 42 carbons; and (b) mixing the active compound and the oil with heat until complete dissolution of the compound.

154-156. (canceled)

157. A topical formulation comprising an active compound and an oil, wherein the formulation is made by a method comprising the following steps: (a) providing an active compound, a solubility enhancing agent and an oil made substantially of straight chain esters of mono-unsaturated long chain fatty acids and fatty alcohols having an average total carbon chain length of about 42 carbons; (b) dissolving the active compound into a solution comprising a solubility enhancing agent; and (c) mixing the solution of step (b) into the oil until complete dissolution of the compound into the solution.

158-160. (canceled)

161. A topical formulation comprising a fine particle dispersion of an active compound in an oil, wherein the formulation is made by a method comprising the following steps: (a) providing an active compound and an oil made substantially of straight chain esters of mono-unsaturated long chain fatty acids and fatty alcohols having an average total carbon chain length of about 42 carbons; (b) mixing the active compound and the oil under high-pressure and accelerating the mixture to high velocities, thereby subjecting the mixture to high shear rates to produce particles of uniform size and making a particle dispersion of the active compound in the oil,
wherein optionally the mixture is accelerated to pressures ranging from about 3,000 to about 23,000 psi.

162-168. (canceled)

169. A method for making a formulation comprising an emulsion of an insoluble compound in a jojoba oil comprising mixing the insoluble compound in a jojoba oil and accelerating the mixture to high velocities, thereby subjecting the mixture to high shear rates to produce an emulsion of uniform droplets,
wherein optionally wherein the mixing is done in a high pressure microfluidizer-homogenizer, and optionally the process pressures range from about 3,000 to about 23,000 psi.

170-177. (canceled)

178. A formulation comprising an emulsion of an insoluble compound in a jojoba oil made by a method comprising mixing the insoluble compound in a jojoba oil and accelerating the mixture to high velocities, thereby subjecting the mixture to high shear rates to produce an emulsion of uniform droplets.

179. A method of making a topical formulation comprising an active compound and jojoba oil comprising the following steps: (a) providing an active compound and a jojoba oil; and (b) mixing the active compound and the jojoba oil at room temperature without the use of heat until complete dissolution of the compound.

180. (canceled)

181. A method of making a topical formulation comprising an active compound and jojoba oil comprising the following steps: (a) providing an active compound and a jojoba oil; and (b) mixing the active compound and the jojoba oil with heat until complete dissolution of the compound.

182-188. (canceled)

189. A method of making a topical formulation comprising a fine particle dispersion of an active compound in jojoba oil comprising the following steps: (a) providing an active compound and a jojoba oil; (b) mixing the active compound and the jojoba oil under high-pressure and accelerating the mixture to high velocities, thereby subjecting the mixture to high shear rates to produce particles of uniform size and making a particle dispersion of the active compound in the jojoba oil.

190-235. (canceled)
